(19) United States
(12) Patent Application Publication
(10) Pub. No.: US 2026/0241160 A1
(43) Pub. Date: Aug. 20, 2026

(54) ELECTRODE, MEDICAL DEVICE, SYSTEM AND METHOD OF MANUFACTURE

(71) Applicant: CAMBRIDGE ENTERPRISE LIMITED, Cambridge, Cambridgeshire (GB)

(72) Inventors: Christopher PROCTOR, Cambridge, Cambridgeshire (GB); Yaw Ansong SENIOR, Cambridge, Cambridgeshire (GB)

(73) Assignee: CAMBRIDGE ENTERPRISE LIMITED, Cambridge, Cambridgeshire (GB)

(21) Appl. No.: 19/102,127

(22) PCT Filed: Aug. 9, 2023
(86) PCT No.: PCT/GB2023/052102

§ 371 Date: Feb. 7, 2025

(30) Foreign Application Priority Data
Aug. 9, 2022 (GB) 2211608.1

(51) Int. Cl. A61N 1/05; A61N 1/378; A61N 1/40

(52) U.S. Cl. CPC A61N 1/0551; A61N 1/3787; A61N 1/40

(57) ABSTRACT

The present invention provides an electrode having a substrate and a conductive polymer film layer on the exposed surface, the film layer having a capacitance per area of at least 0.5 F/cm² and/or wherein the film layer has a thickness of between 50-1000 μm. A medical device having a plurality of electrodes wherein at least one of the electrodes includes a polymer film layer, the film layer having a capacitance per area of at least 0.5 F/cm² is also provided, as are systems including the medical device, methods of treatment of a spinal cord lesion and methods of manufacturing the electrodes.

The present invention relates to electrodes, medical devices, systems and methods of treatment and manufacture. The invention is of particular, but not exclusive, relevance to implantable devices, for example for treating spinal cord injury.

Spinal cord injury (SCI) is a devastating condition affecting over 1.5 million people globally, including between 250,000 and 500,000 additional people every year. SCI occurs when the neuronal tracts of the spinal cord are damaged and leads to a permanent loss of sensation, motor and organ control below the lesion.

SCI also leads to a socioeconomic burden for the individual, their families and the healthcare system in supporting them.

At present there are no treatments used which are directed at treating the primary injury. Treatments are therefore typically limited to treating secondary damage, for example by performing decompressive surgery followed by rehabilitation therapy to encourage tissue growth to restore some of the lost function.

Electrical stimulation of neural tissue using implantable electrodes has shown promise as a treatment for SCI. Electric fields applied over neurons with damaged tracts are well known to encourage and guide the regeneration of new axons. When DC electric fields are applied to the spinal cord for a period of weeks following injury (known as OFS—oscillating field stimulation), recovery of neurological deficits has been observed in both animal models and human SCI patients.

However, translation of OFS technology into clinical use has stalled in the last decade. This is largely due to the lack of an adequate implantable platform for delivering electric fields to the spinal cord tissue. Previous OFS tests have relied on the use of re-purposed pacemaker leads, which are bulky, require additional surgery to explant both the leads and battery after delivery of treatment, and can cause secondary damage to sensitive neural tissues. At least one reason for this is that the capacitance of the implantable electrodes is low and therefore several electrodes are required to provide the necessary electrical field at the implantation site.

The present invention aims to solve one or more of the above problems.

Aspects of the present invention aim to provide devices and electrodes for devices that can deliver OFS without invasive surgery, or with minimally-invasive surgery.

Aspects of the present invention aim to provide methods of treating spinal cord injury using electrical stimulation which are enabled by new devices.

At their most general, aspects of the present invention provide electrodes and devices incorporating electrodes which have a high capacitance in particular in order to deliver sustainable electrical field therapy at low and ultra-low frequencies. Further aspects relate to methods of manufacturing such electrodes and to methods of treatment using electrical fields.

A first aspect of the present invention provides a medical device having a plurality of electrodes wherein at least one of the electrodes includes a polymer film layer, the film layer having a capacitance per area of at least 0.1 F/cm².

Preferably the film layer has a capacitance per area of at least 0.5 F/cm², preferably 1 F/cm², more preferably at least 5 F/cm². In some embodiments the film layer may have a capacitance of up to 10 F/cm².

A capacitance of 0.1 F/cm² corresponds to a charge storage capacity of 100 mC/cm² over a standard cyclic voltammetry range of 1 volt. Similar correspondences arise for the other capacitance values given herein, for example a capacitance of 0.5 F/cm² corresponds to a charge storage capacity of 500 mC/cm² over a standard cyclic voltammetry range of 1 volt.

Preferably the capacitance of the film layer is sufficient to sustain a non-faradaic current density between the electrodes of at least 100 μA/cm² for a period of at least 100 seconds.

Preferably the film layer has a thickness of between 50-1000 μm. More preferably the film layer has a thickness of over 100 μm and more preferably over 250 μm. The thickness of the film layer can increase the capacitance.

The device of this aspect can generate electric fields for therapeutic purposes. For example, in some embodiments the electric fields can be used in OFS to direct nerve tissue regeneration across a lesion site of a spinal injury. The device aims to provide a uniform electric field across the lesion while minimizing the surgical footprint.

In preferred embodiments the film layer is formed from PEDOT/PSS, which is a biocompatible conductive polymer.

The conductive polymer layer may be attached to a conductive surface which could take many forms such as: one or more thin wires; a thin metal foil (1-50 μm); a wire mesh; a thin metal film (<1 μm); or metallic particles or flakes embedded in the polymer film. However, in certain embodiments the polymer layer itself may be conductive enough to preclude the need for a conductive surface.

Preferably the device is flexible. In certain embodiments the flexibility of the device allows it to conform to a spinal cord region or other region of the body in which it is to be implanted. The flexibility may also aid in a minimally invasive explanation procedure wherein the electrodes are removed through a small incision.

In most embodiments one side of the electrode would be insulated such that current is primarily passed through the exposed conductive polymer layer.

In particular embodiments the device may have a bend radius of no more than about 2 mm, preferably no more than 1.5 mm and more preferably no more than 0.5 mm. Bend radius, which is measured to the inside curvature, is the minimum radius that a component (in this case the device) can be bent in at least one direction without damaging it. The bend radius as defined here refers to elastic deformation as opposed to plastic deformation such that a device bent under an applied force to a radius greater than the minimum bend radius would return at least part way to its original shape with the removal of the applied force. In other words, in these embodiments, the device of this aspect can be bent to an inside curvature of 2 mm, for example by rolling when the device is being arranged for insertion into a patient, and subsequently deployed (e.g. unrolled) to an expanded, less bent configuration (e.g. a substantially planar configuration) and still function exactly as it did prior to bending. Alternatively, the device may be rolled or curved in implanted form in order to conform to the region of implantation or curved to match the surface of the skin for a wearable application.

The device may further include a pulse generator which is coupled to the electrodes and arranged to control the electrical field between the electrodes. The pulse generator may be coupled to the electrodes by wires or may be coupled wirelessly by any known communication protocol.

Preferably the pulse generator is arranged to ensure that the voltages between the electrodes do not exceed 1.2V. 1.2V is the “water window” and voltages above the “water window” can lead to water splitting as well as other Faradaic reactions that can potentially create toxic by-products.

The pulse generator may be arranged to cause the voltage between the electrodes to reverse polarity with a period of at least 300 seconds. A long period allows for axon regrowth to occur in the direction of the polarity before reversal to stimulate axon regrowth from the other side of the lesion. Typically OFS oscillatory periods are in range of 2-30 minutes, more often 10-15 minutes.

In certain embodiments the device has an overall thickness of less than 1.5 mm and/or a width of less than 2 cm. The device is thus very small and therefore suitable for implantation, for example through a percutaneous incision.

In certain embodiments the device is an implantable device. Implantable devices of these embodiments are preferably configured for implantation and/or explantation through an incision of no more than 1 cm, thus being usable in minimally-invasive surgery such as percutaneous surgery.

The device is preferably configured to wirelessly receive electrical power from a separate power source. The wireless transmission of power to implanted devices is well-known and the device of this aspect can make use of any known technology for this purpose. Wireless transmission of power allows the power source to be positioned outside of the patient when the device is implanted, and therefore the power source can be larger than would be the case for an implantable power source, and can also be easily changed and/or recharged.

A further aspect of the present invention provides a system for treating spinal cord injury, the system including: a medical device according to the above first aspect, including some, all or none of the optional and preferred features of that aspect, wherein the device is an implantable device; and a pulse generator coupled to the electrodes of the device and arranged to create a varying electrical field between the electrodes.

Preferably the pulse generator is also implantable.

The pulse generator may be coupled to the electrodes by wires or may be coupled wirelessly by any known communication protocol. Where the pulse generator is wirelessly coupled it need not be implantable (but in certain embodiments is implantable).

The system may further include a power source. Preferably the power source is wirelessly coupled to the pulse generator. This allows the power source to be positioned outside of the patient in configurations in which the pulse generator is implanted, and therefore the power source can be larger than would be the case for an implantable power source, and can also be easily changed and/or recharged.

Preferably the pulse generator is arranged to ensure that the voltages between the electrodes do not exceed 1.2V. 1.2V is the “water window” and voltages above the “water window” can lead to water splitting as well as other Faradaic reactions that can potentially create toxic by-products.

The pulse generator may be arranged to cause the voltage between the electrodes to reverse polarity with a period of at least 300 seconds. A long period allows for axon regrowth to occur in the direction of the polarity before reversal to stimulate axon regrowth from the other side of the lesion. Typically OFS oscillatory periods are in range of 2-30 minutes, more often 10-15 minutes.

A further aspect of the present invention provides a method of treating a spinal cord lesion in a patient, the method including the steps of: percutaneously implanting a plurality of electrodes to positions proximate the spinal cord of the patient and on opposing sides of the lesion; applying an electrical field between the electrodes to stimulate axon regrowth; and periodically reversing the polarity of the electrical field to stimulate axon regrowth from the other side of the lesion.

The electrodes which are implanted may be electrodes according to the aspects described further below, or part of a device according to the above-described first aspect, including some, all or none of the optional and preferred features of either, but need not be either of these.

The step of implanting is preferably performed during decompression surgery, which is a known approach for treating the secondary effects of a spinal injury.

Preferably the method further includes the step of percutaneously explanting the electrodes after the treatment.

A further aspect of the present invention provides an electrode having a substrate and a conductive polymer film layer on the exposed surface, the film layer having a capacitance per area of at least 0.1 F/cm².

Preferably the film layer has a capacitance per area of at least 0.5 F/cm², preferably at least 1 F/cm², more preferably at least 5 F/cm². In some embodiments the film layer may have a capacitance of up to 10 F/cm².

A further aspect of the present invention provides an electrode having a substrate and a conductive polymer film layer on the exposed surface, the film layer having a thickness of between 50-1000 μm.

Preferably the film layer has a thickness of over 100 μm and more preferably over 250 μm.

In both of these aspects, the electrode preferably has a thickness of no more than 1.5 mm and width of no more than 1.5 cm. This allows the electrode, along with any associated components to be implanted, for example percutaneously, by a minimally-invasive surgical method.

The electrodes of these aspects may be used as the electrodes of the medical device of the above described first aspect.

A further aspect of the present invention provides a method of manufacturing an electrode, the method including the steps of: coating a surface of a conductive substrate with a conductive polymer film layer using a solution of said conductive polymer whilst soft-baking the substrate and film layer, until the film layer reaches a thickness of at least 50 μm; and hard-baking the resulting structure.

The electrode formed by this method may be an electrode according to the above described aspects, including some, all or none of the optional and preferred features of those aspects, but need not be.

The method may further include the step of coating the surface of the substrate onto which the conductive polymer film layer is coated with an adhesion promoting layer such as a hydrophilic polymer like PVA prior to coating with the conductive polymer film layer.

According to a further aspect of the invention, there is provided a method of treating a human or animal body, the method comprising implanting a medical device or electrode according to the above-described aspects.

Unless indicated otherwise, any of the features (including the optional or preferred features) described in relation to one of the above aspects are equally applicable in combination with the devices, systems and methods of any of the other above-described aspects.

The invention is described below, by way of example, with reference to the accompanying figures in which:

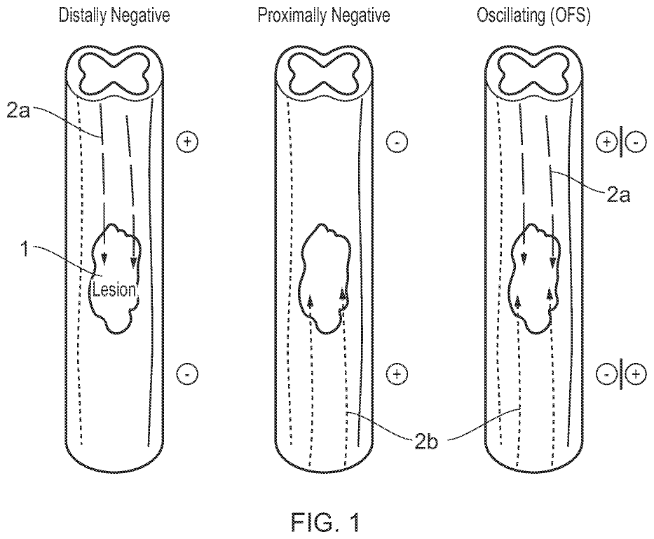

FIG. 1 is an illustration of the operating principle of OFS;

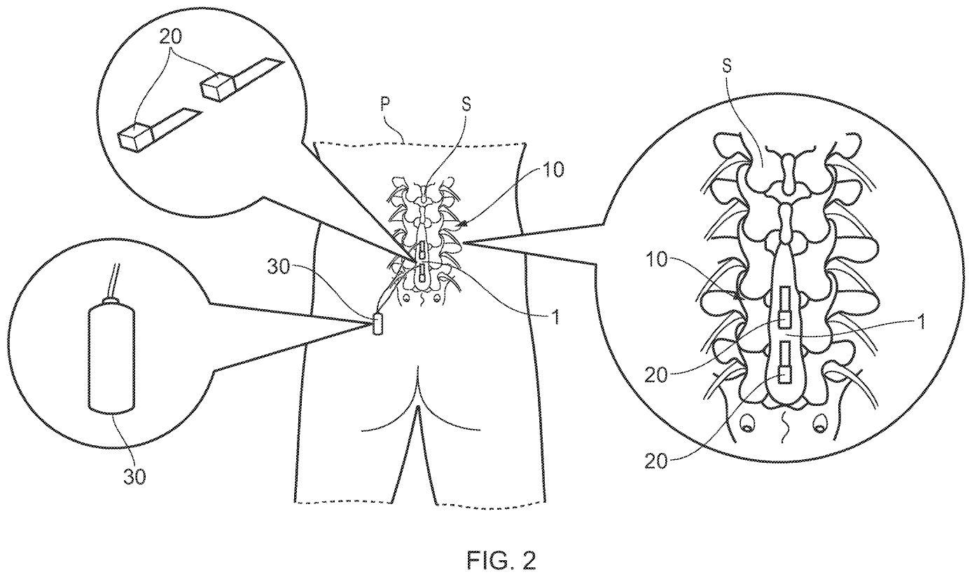

FIG. 2 illustrates the use of implanted electrodes to deliver OFS according to an embodiment of the present invention;

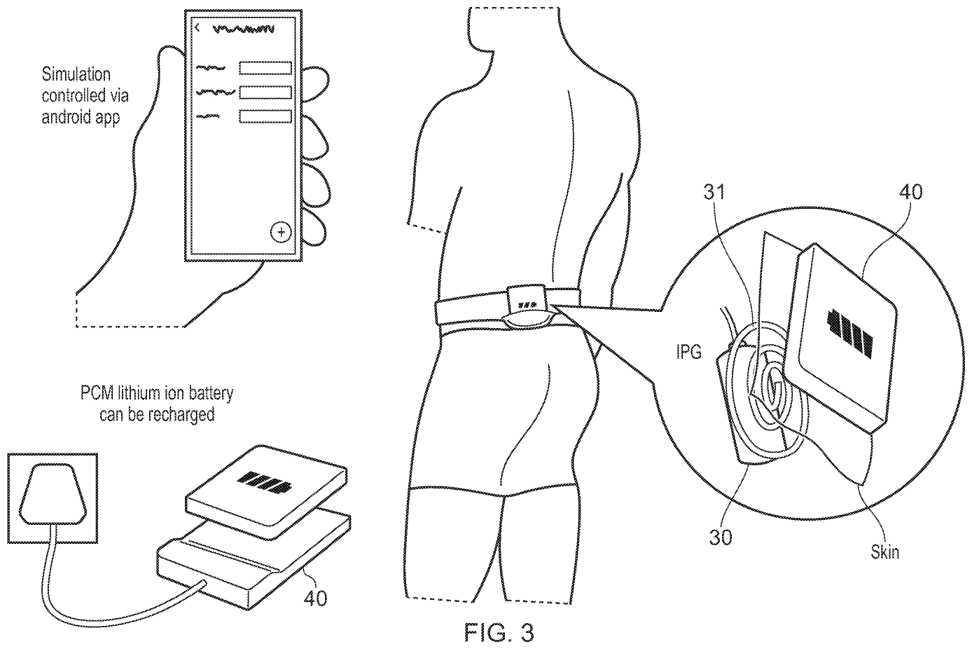

FIG. 3 illustrates a system according to an embodiment of the present invention;

FIG. 3 shows a device according to an embodiment of the present invention;

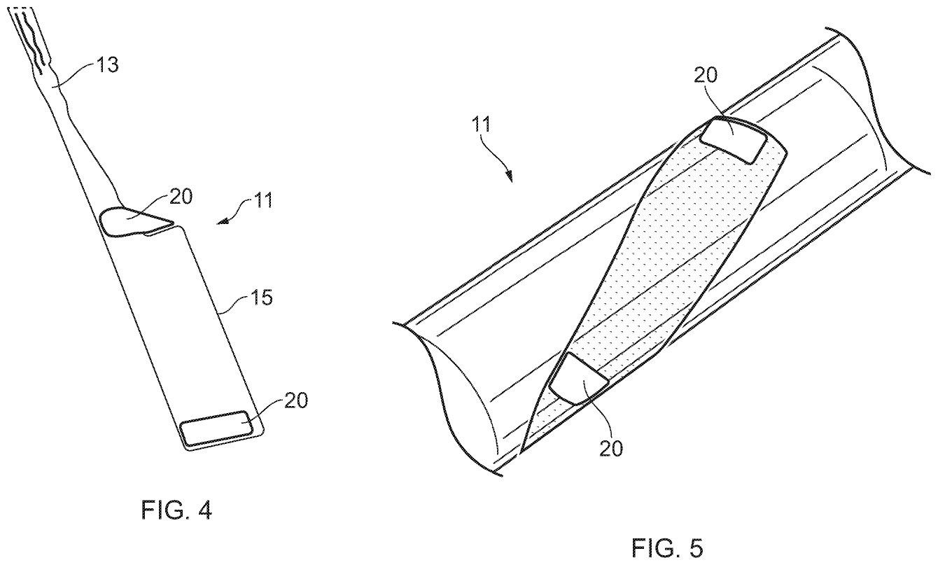

FIG. 4 shows the device of FIG. 3 arranged to conform to a curved surface of similar dimensions to a spinal cord;

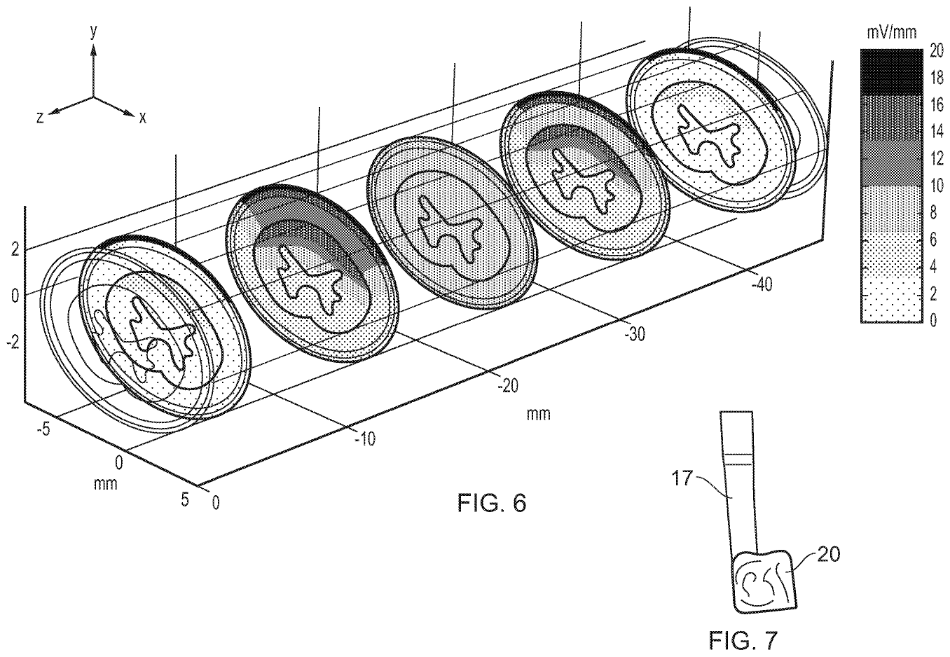

FIG. 6 shows the results of a simulation of the electric potential in spinal cord tissues generated by a device according to an embodiment of the present invention;

FIG. 7 shows a single electrode according to an embodiment of the present invention;

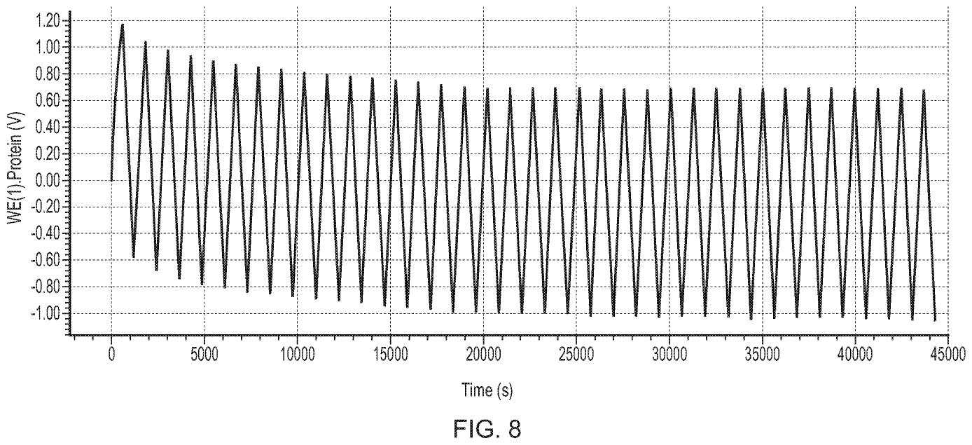

FIG. 8 is a potentiometry graph showing approximately 12 hours use of a device according to the present invention in an in vitro test;

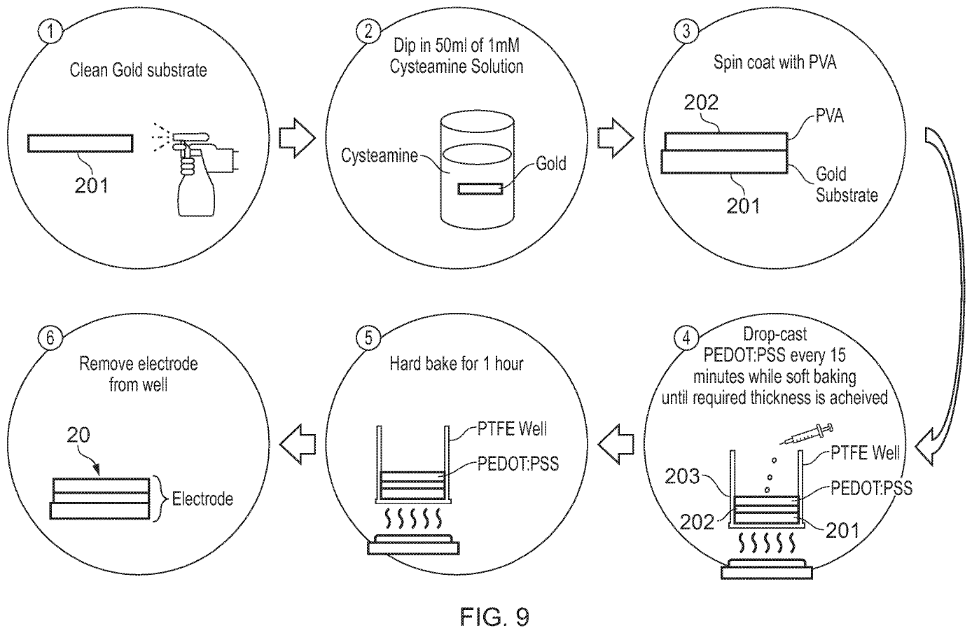

FIG. 9 is a schematic showing the steps in the manufacture of an electrode according to an embodiment of the present invention; and

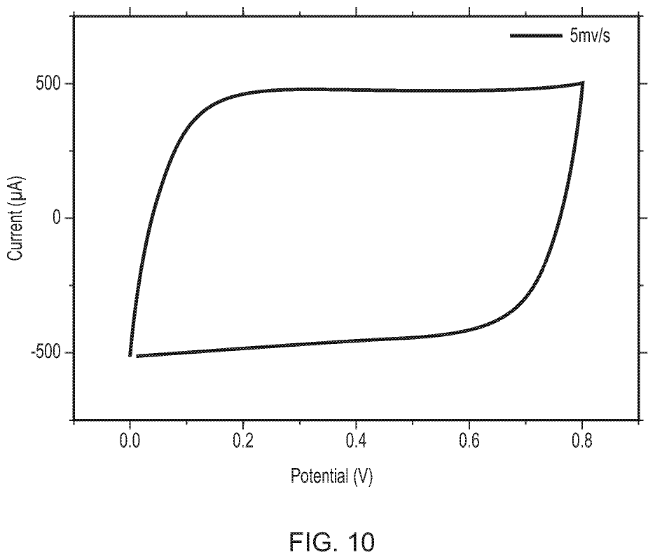

FIG. 10 is a cyclic voltammogram of an electrode according to an embodiment of the present invention.

Embodiments of the present invention are described below in connection with the treatment of SCI using OFS. However, it will be appreciated that this is only one use to which electrodes, devices and systems according to embodiments of the present invention can be put. In other embodiments, which are not described in detail here, electrodes, devices and systems according to embodiments of the present invention could, for example, be used in wearable applications, for example in wound healing.

FIG. 1, which is taken from (1), illustrates the principles on which OFS operates. A continuous electrical field enhances regrowth of damaged axons 2a, 2b across the lesion 1. Growth is stimulated towards the negative pole. Therefore, in OFS, the polarity of the applied electric field periodically switches to allow for axons 2b below the lesion 1 to be directed cranially towards the brain to recover sensory perception, and axons 2a above the lesion 1 to be directed caudally towards the periphery to recover motor control, in an alternating fashion.

The electric field in OFS is typically maintained for several minutes (2-30 minutes, more often 10-15 minutes) in each direction between the periodic switches of polarity, requiring electrodes with high capacitance. The original proposed OFS systems attempted to achieve this by using a collection of six relatively large pacemaker electrodes. However, the large surgical footprint of these pacemaker leads increased risk for patients and provided a suboptimal electric field distribution across the lesion.

Embodiments of the present invention provide methods of delivering OFS and devices and systems which can deliver OFS which are implantable through minimally-invasive surgery, for example percutaneously.

FIG. 2 illustrates, schematically, components of a system 10 for treating SCI according to an embodiment of the present invention. The system 10 includes two implantable electrodes 20 which, in use, are implanted in the spinal cord region S of the patient P by laminectomy and arranged approximately equidistant either side of a known lesion site 1. Examples of electrodes 20 according to further embodiments of the present invention are described in more detail below.

The electrodes 20 are connected or coupled to an implantable pulse generator (“IPG”) 30. Whilst the connections in FIG. 2 are shown as wired connections, wireless connection by any known means is also possible. Similarly, whilst the IPG 30 in FIG. 2 is shown implanted in the body of the patient P it may also be located outside the body. The IPG 30 may be implanted at a position which is proximate, but not directly in or adjacent to, the spinal cord region S, for example the gluteal region. This means that the IPG 30, whilst preferably small to allow implantation without significant surgery, need not be of a scale, shape or material which is itself suitable for implantation in the spinal cord.

An example of an IPG 30 is a pulse generator chip from Teliatry, Inc. of Richardson, Texas which is substantially cuboid and glass-encapsulated, with dimensions of 11 mm×7 mm×1.5 mm. This is small enough to be implanted, and explanted, through minimally invasive procedures.

FIG. 3 illustrates a system according to an embodiment of the present invention. The implanted components of the system are as discussed above in relation to FIG. 2. The system also includes a controller 40 with an external PCM lithium ion rechargeable battery pack and induction coil 31 implanted below the skin of the patient to wirelessly power the implanted components by known wireless power transfer methods. The system can also include a wireless device, such as a mobile handset 50 which can control the stimulation via an application running on the device and sending instructions to the controller 40.

Advances in microfabrication technologies and material sciences have given rise to a new generation of neuro-implantable technologies (2, 3). Devices according to embodiments of the invention comprise ultra-flexible parylene sheets capable of conforming to the surface of neural organs combined with conducting polymer electrodes capable of delivering higher currents than their traditional metal counterparts in a fraction of the space.

By adapting thin-film conducting polymer implant technology to the requirements and properties of the injured spinal cord, the devices can be used to more effectively deliver OFS therapy to SCI patients, inducing regeneration following injury and improving their recovery. In embodiments of the invention, the devices and systems are implanted at the time of decompression surgery.

FIG. 4 illustrates a device 11 according to an embodiment of the present invention. The device has a thin flexible parylene substrate 15 with two polymer electrodes 20 formed spaced apart on the substrate. An extension 13 of the substrate may provide for connectivity to a wireless power source and/or controller (for example IPG 30 as described above), or may provide conductors for a wired connection to the power source and/or controller.

FIG. 5 illustrates a device 11 such as that shown in FIG. 4 which has been rolled around a tube of 1 cm diameter, demonstrating how the device 11 can flexibly conform to a curved surface of similar dimensions to the spinal cord and the electrodes 20 can be positioned such that they are axially spaced along the spinal cord. In order to conform to the spinal cord of a patient in this manner, the device 11, or at least the portion of the device 11 on which the electrodes 20 are mounted, preferably has a bend radius of 2 mm or less.

The electrodes 20 are coated with the conducting polymer PEDOT: PSS which offers a 100-fold increase in electrode capacitance for the same surface area compared to metal electrodes (4, 5). PEDOT: PSS coatings have previously been demonstrated to be biocompatible. The capacitance of the polymer electrodes 20 has been further increased by creating thicker electrodes as discussed further below.

The combination of the high capacitance PEDOT: PSS electrodes 20 and a thin flexible substrate 15 that can conform to the shape of the spinal cord enables the devices according to embodiments of the invention to maintain a uniform electric field across a lesion thereby providing optimal conditions for directed axon regeneration.

A computational model of the system illustrated in FIGS. 2 and 3 using a device as shown in FIGS. 4 and 5 has been developed. FIG. 6 shows the model of the electrical field distribution in the simulated spinal cord between the two electrodes. The simulation shows that two epidurally-placed high capacitive polymer electrodes can maintain a uniform electric field across the spinal cord. The magnitude of the electric field, in the range of 10 m V/mm, is known to be sufficient for directing cellular growth.

In the embodiment described above in relation to FIGS. 4 and 5, the device had two electrodes 20 mounted on a single substrate 17. However, in alternative embodiments of the invention, the electrodes 20 are separate and formed individually on their own substrates. FIG. 7 shows an example of such an electrode 20. As with the device illustrated in FIGS. 3 and 4, the device shown in FIG. 7 may have further components formed on the substrate 17 which may provide for connectivity to a wireless power source and/or controller (for example IPG 30 as described above), or may provide conductors for a wired connection to the power source and/or controller.

FIG. 8 shows the use of a device according to the present invention in an in vitro test to demonstrate the safe and stable electrical fields that can be achieved. The graph shows an OFS-type variation with a period of approximately 15 minutes. The electrodes in the test had PEDOT:PSS layers of approximately 250 μm thickness. FIG. 8 shows that the electrodes are able to maintain the desired field (600 μA), whilst also keeping the voltage below the “water window” of ~1.2 V. Potentials above the “water window” can lead to water splitting as well as other Faradaic reactions that can potentially create toxic by-products. The operation of the device within the desired ranges thus reduces the risk of damage to surrounding neural tissue when the device is implanted in the spinal cord region of a patient.

FIG. 9 illustrates a method of manufacturing an electrode according to an embodiment of the present invention. The fabrication starts with a cleaned gold substrate 201. The gold substrate is dipped in 50 mL of 1 mM Cysteamine solution before being spin coated with a PVA layer 202 on one surface.

A PTFE wall is formed around the prepared substrate to allow drop casting of PEDOT:PSS to create a layer of PEDOT:PSS 203 on the PVA layer 202. Further amounts of PEDOT:PSS are added every 15 minutes whilst the electrode is soft baked until a desired thickness is reached. Soft-baking is a process by which the electrode is heated at a temperature (and over a time period) which partially but not fully dries the film. For the thick PEDOT:PSS films such as used in the electrodes described above, such soft-baking is typically in the range of 100° C. for 10 minutes. Typically electrodes of embodiments of the invention have thicknesses between 50-500 μm.

Once the desired thickness of PEDOT:PSS has been reached, the electrode is hard baked for 1 hour and then the composite electrode 20 is removed from the PTFE well. Compared to soft-baking, hard-baking is heating at a temperature (usually but not always higher than the soft-baking temperature) for a time sufficient to completely dry the film and complete any thermally driven reactions such as cross-linking. In the method shown, the hard-baking includes heating at 130° C. for 1 or more hours.

Drop casting PEDOT:PSS allows layers of much greater thickness to be achieved than using current approaches of electrochemical deposition or solution-based spin coating. The present inventors have found that, contrary to existing beliefs, increased thickness of PEDOT:PSS can produce electrodes with correspondingly increased capacitance (in particular, without saturation in the increase of capacitance, which was previously expected once thicknesses significantly exceeded 1 μm) and without the need to change other characteristics of the PEDOT:PSS layer such as the porosity.

FIG. 10 is a cyclic voltammogram of an electrode according to the present invention, manufactured in accordance with the process described above with reference to FIG. 9. The electrode has a PEDOT:PSS film with a thickness of 200 μm and an area of 0.25 mm² for a volume of 0.05 mm³.

The voltammogram uses a scan rate of 5 mV/s between 0.0-0.8V and shows that a charge storage capacity of 521 mC/cm² is achieved. This corresponds to a capacitance of 0.65 F/cm².

The forgoing description is exemplary in nature only, and the skilled person will understand that changes and variations on the disclosed embodiments are possible within the scope of the claims. The claims define the invention.

### REFERENCES

(1) Li, Jianming, Weak direct current (DC) electric fields as a therapy for spinal cord injuries: review and advancement of the oscillating field stimulator (OFS), Neurosurgical Review, 42, 825-834 (2019). https://doi.org/10.1007/s10143-018-01068-y
(2) Someya, T. ; Bao, Z., Malliaras, G. G. The Rise of Plastic Bioelectronics. Nature 2016, 540 (7633), 379-385. https://doi.org/10.1038/nature 21004.
(3) Bettinger, C. J. ; Ecker, M. ; Kozai, T. D. Y. ; Malliaras, G. G. ; Meng, E. ; Voit, W. Recent Advances in Neural Interfaces-Materials Chemistry to Clinical Translation. MRS Bulletin 2020, 45 (8), 655-668. https://doi.org/10.1557/mrs.2020.195.
(4) Proctor, C. M. ; Rivnay, J. ; Malliaras, G. G. Understanding Volumetric Capacitance in Conducting Polymers. Journal of Polymer Science Part B: Polymer Physics 2016, 54 (15), 1433-1436. https://doi.org/10.1002/polb.24038.
(5) Rivnay, J. ; Leleux, P. ; Ferro, M. ; Sessolo, M. ; Williamson, A. ; Koutsouras, D. A. ; Khodagholy, D. ; Ramuz, M. ; Strakosas, X. ; Owens, R. M. ; Benar, C. ; Badier, J.-M. ; Bernard, C. ; Malliaras, G. G. High-Performance Transistors for Bioelectronics through Tuning of Channel Thickness. Science Advances 2015, 1 (4), e 1400251. https://doi.org/10.1126/sciadv.1400251.

All of the above references are hereby incorporated by reference in their entirety.

1. A medical device having a plurality of electrodes wherein at least one of the electrodes includes a polymer film layer, the film layer having a capacitance per area of at least 0.5 F/cm².

2. A medical device according to claim 1 wherein the film layer has a thickness of between 50-1000 μm.

3. A medical device according to claim 1 wherein the film layer is formed from PEDOT/PSS.

4. A medical device according to claim 1 wherein the device is flexible.

5. A medical device according to claim 1 further including a pulse generator which is coupled to the electrodes and arranged to control the electrical field between the electrodes.

6. A medical device according to claim 1 wherein the device has an overall thickness of less than 1.5 mm and/or a width of less than 2 cm.

7. A medical device according to claim 1 wherein the device is an implantable device and is preferably configured for implantation and/or explantation through an incision of no more than 1 cm.

8. A medical device according to claim 1 wherein the device is configured to wirelessly receive electrical power from a separate power source.

9. A system for treating spinal cord injury, the system including:
a medical device according to claim 1, wherein the device is an implantable device; and
a pulse generator coupled to the electrodes of the device and arranged to create a varying electrical field between the electrodes.

10. A system according to claim 9, wherein the pulse generator is implantable.

11. A system according to claim 9, further including a power source which is wirelessly coupled to the pulse generator.

12. A system according to claim 9 wherein the pulse generator is wirelessly coupled to the electrodes.

13. A system according to claim 9 wherein the pulse generator is arranged to ensure that the voltages between the electrodes do not exceed 1.2V.

14. A system according to claim 9 wherein the pulse generator is arranged to cause the voltage between the electrodes to reverse polarity with a period of at least 300 seconds.

15. A method of treating a spinal cord lesion in a patient, the method including the steps of:
percutaneously implanting a plurality of electrodes to positions proximate the spinal cord of the patient and on opposing sides of the lesion;
applying an electrical field between the electrodes to stimulate axon regrowth; and
periodically reversing the polarity of the electrical field to stimulate axon regrowth from the other side of the lesion.

16. A method according to claim 15 wherein the step of implanting is performed during decompression surgery.

17. A method according to claim 15 further including the step of percutaneously explanting the electrodes after the treatment.

18. An electrode having a substrate and a conductive polymer film layer on the exposed surface, the film layer having a capacitance per area of at least 0.5 F/cm².

19. An electrode having a substrate and a conductive polymer film layer on the exposed surface, the film layer having a thickness of between 50-1000 μm.

20. An electrode according to claim 18 wherein the electrode has a thickness of no more than 1.5 mm and width of no more than 1.5 cm.

21. A method of manufacturing an electrode, the method including the steps of:
coating a surface of a conductive substrate with a conductive polymer film layer using a solution of said conductive polymer whilst soft-baking the substrate and film layer, until the film layer reaches a thickness of at least 50 μm; and
hard-baking the resulting structure.

22. A method according to claim 21 further including the step of coating the surface of the substrate onto which the conductive polymer film layer is coated with an adhesion promoting layer prior to coating with the conductive polymer film layer.

23. An electrode according to claim 19 wherein the electrode has a thickness of no more than 1.5 mm and width of no more than 1.5 cm.
